(19) United States
(12) Patent Application Publication
(10) Pub. No.: US 2010/0197783 A1
(43) Pub. Date: Aug. 5, 2010

(54) Radiation Protection Using Single Wall Carbon Nanotube Derivatives

(76) Inventors: James M. Tour, Bellaire, TX (US); Meng Lu, Houston, TX (US); Rebecca Lucente-Schultz, Houston, TX (US); Ashley Leonard, Houston, TX (US); Condell Dewayne Doyle, Nocona, TX (US); Dmitry V. Kosynkin, Houston, TX (US); Brandi Katherine Price, Houston, TX (US)

Correspondence Address: WINSTEAD PC, P.O. BOX 50784, DALLAS, TX, 75201, US

(21) Appl. No.: 12/593,585

(22) PCT Filed: Mar. 26, 2008
(86) PCT No.: PCT/US08/58268

§ 371 Date: Mar. 26, 2010

Related U.S. Application Data
(60) Provisional application No. 60/908,115, filed on Mar. 26, 2007.

(51) Int. Cl. A61K 31/215; A61P 43/00; C07C 229/00

(52) U.S. Cl. 514/530; 562/442; 977/750; 977/752; 977/742

(57) ABSTRACT

A method of reducing side effects of damage in a human subject exposed to radiation includes administering to the human subject carbon nanotubes in a pharmaceutically acceptable carrier after or prior to exposure to radiation. A composition for reducing radical damage includes a carbon nanotube which is functionalized (1) for substantial water solubility and (2) with a radical trapping agent appended to the carbon nanotube forming a radical scavenger-carbon nanotube conjugate.

### CROSS-REFERENCE TO RELATED APPLICATIONS

This application claims priority U.S. Provisional Patent Application No. 60/908,115 filed Mar. 26, 2007.

### BACKGROUND

A variety of cellular oxidative stresses can lead to the generation of potentially damaging radical species. Oxidative stress is caused by an imbalance between the production of reactive oxygen and a biological system's ability to detoxify the reactive intermediates or easily repair the resulting damage. The cellular redox environment is typically preserved by enzymes that maintain a reduced state through a constant input of metabolic energy. Disturbances in this normal redox state can cause toxic effects through the production of peroxides and free radicals that damage all components of the cell, including proteins, lipids, and DNA.

In humans, oxidative stress is involved in many diseases, such as atherosclerosis, Parkinson's disease and Alzheimer's disease. External environmental conditions may also be responsible for the formation of damaging radical species, such as exposure to radiation. It would be beneficial, therefore, to provide compositions and methods that can quench such radical species in order to ameliorate the harmful effects of these radicals.

### SUMMARY

In some aspects, the present disclosure provides a method of reducing side effects of radical damage in a human subject exposed to radiation which includes administering to the human subject carbon nanotubes in a pharmaceutically acceptable carrier.

The present disclosure provides a composition which includes, but is not limited to a nanostructured material, which may be functionalized to confer substantial water solubility; and a radical trapping agent appended to this nanostructured material to form a radical scavenger-nanostructure conjugate.

In other aspects, the present disclosure provides a formulation which includes a functionalized nanostructured material which can be a single-wall carbon nanotube (SWNT), double-wall carbon nanotube (DWNT) and multi-wall carbon nanotube (MWNT) (where there are three or more walls predominating in a sample), any of which is functionalized for water solubility and also is useful for quenching free radicals in biological systems.

The foregoing has outlined the features and technical advantages in order that the detailed description that follows may be better understood. Additional features and advantages will be described hereinafter which form the subject of the claims.

### BRIEF DESCRIPTION OF THE DRAWINGS

The foregoing and other features and aspects of the present invention will be best understood with reference to the following detailed description of a specific embodiment of the invention, when read in conjunction with the accompanying drawings, wherein:

FIG. 1 shows a hydrogel useful for the delivery of carbon nanotubes by oral administration.

FIG. 2 shows an overview of the oxygen radical absorbance capacity (ORAC) assay.

FIG. 3 shows a comparison of TROLOX® Equivalents obtained for each of the compounds 12, 13, 15, 16 and 17 relative to the known fullerene derivative DF-1 using the ORAC assay.

FIG. 4 shows an in vitro assay, for assessing the radiation protection and mitigation effects of compounds 16 and 17, using rat small intestine crypt cells (ATCC cat #CRL-1592).

FIGS. 5A-5C show normal zebrafish growth. The normal growth of zebrafish 28 hours post-fertilization (FIG. 5A), 2 days post-fertilization (FIG. 5B), and 4 days post-fertilization (FIG. 5C) are depicted. The spherical structures in 5A and 5B are the yolk sacs.

FIG. 6 shows a schematic of a radiation protection assay in vivo in zebrafish using these nanotube compounds.

FIG. 7 shows a schematic of a radiation mitigation assay in vivo in zebrafish using these nanotube compounds.

FIG. 8 shows grading “curly up” in zebrafish in response to exposure to radiation. The more severe the damage, the greater the “curly up” angle.

FIGS. 9A-9E show radiation protection effects of compound 16 in zebrafish.

FIG. 9A shows degree of “curly up” in 4 days post-fertilization (DPF) zebrafish exposed to radiation and FIG. 9B depicts degree of “curly up” in zebrafish injected with compound 16 exposed to radiation. FIGS. 9C-9D depict, degree of “curly up” in zebrafish, 6 days post-fertilization, exposed to radiation alone (FIG. 9C) or injected with compound 16 and subsequently exposed to radiation (FIG. 9D), respectively. FIG. 9E shows a normal zebrafish not subject to radiation.

FIG. 10 shows radiation protection and mitigation data in zebrafish injected with compound 16 before radiation exposure (protection) or administering compound 16 following radiation exposure (mitigation).

FIG. 11 shows an assessment of radiation protection in vivo in a mouse model by evaluating viability of crypt stem cells in the jejunum of mice injected with compound 13 and then exposed to radiation (protection).

### DETAILED DESCRIPTION

The present disclosure provides a method of reducing side effects of radical damage in a human subject or individual exposed to therapeutic or accidental radiation that includes administering to the person a carbon nanotube in a pharmaceutically acceptable carrier after radiation exposure. Side effects of radiation include damage to the intestinal tract lining resulting in nausea, bloody vomiting and diarrhea. Gastrointestinal symptoms of radiation exposure may occur when a victim's exposure is 2 Gy or more but are most severe and may require medical intervention when acute radiation doses to the abdomen or whole body exceed 8-10 Gy at relatively high dose rates at or near 1 Gy/min. Radiation begins to destroy the cells in the body that divide rapidly, including blood, GI tract, reproductive and hair cells. Furthermore, the DNA and RNA of surviving cells may be damaged and more susceptible to carcinogenesis.

In alternate embodiments, ameliorating the effects of exposure to radical damage may include processes involving other oxidative stresses to the body not involving radiation exposure. Without being bound by theory, a radical scavenger may operate by reducing the number of free radicals within or nearby a organelle, cell, tissue, organ, or living organism which would reduce the risk of damage to DNA and other cellular components (i.e., RNA, mitochondria, membranes, etc.) that can lead to chronic and/or acute pathologies, including but not limited to cancer, cardiovascular disease, immunosuppression, and disorders of the central nervous system.

The human subject may be a patient of a physician or radiologist performing targeted radiotherapy on the patient, for example. The human subject may also be treated by a first responder in the case of a nuclear disaster, for example. In yet other embodiments, the human subject may self-administer the carbon nanotubes. In these latter two cases, the carbon nanotubes in a pharmaceutically acceptable carrier may be packaged in kit form as part of a first aid kit, for example. This may be useful in laboratories that utilize radioactive materials, in nuclear power plants, or in ambulances, in the case of first responders.

Administration after radiation exposure (termed here mitigation) may be useful as an antidote of sorts in the event of accidental radiation exposure in a laboratory, solar flares in space exploration, therapeutic administration after radiation treatment for cancer, nuclear plant accidents, nuclear or other radiological bombs, exposure in terrorist situations where radiation is present or the like. In other embodiments, a method of reducing side effects of radical damage in a human subject exposed to radiation includes administering to the human subject a carbon nanotube in a pharmaceutically acceptable carrier prior to radiation exposure (termed here as protection) wherein the nanotube material is serving as a prophylactic. Such administration may be planned as part of a radiation treatment regimen for the treatment of cancer, for example, to protect the exposed portions of the human subject's body, for space travel where radiation exposure is anticipated, for first-responders or clean-up teams to nuclear fallout or other radiation-contaminated sites. It has been demonstrated herein that carbon nanotubes and various derivatives show an unusually high radical scavenging ability, which may prove efficacious in protecting living systems from radical-induced decay whether administered before (protection) or after (mitigation) radiation exposure.

The modes of administration may include, without limitation, localized subcutaneous injection and systemically either orally or by injection. Oral administration is of particular interest due to the dire consequences of depletion of crypt cells in the intestinal lining upon general radiation exposure and because of the ease of administration to the general populace not requiring hospitalization or advanced medical assistance. In the event of a nuclear disaster, for example, anything to ease and hasten the process of triage and treatment would be highly desirable. Oral administration of the proposed carbon nanotubes would contribute favorably to this cause. In one embodiment, the carrier vehicle for delivery of the carbon nanotubes is a pH-sensitive mucoadhesive hydrogel for the oral administration of carbon nanotubes. Oral administration of the proposed carbon nanotubes may be possible through the use of specialized hydrogels, for example.

A hydrogel carrier may serve to protect the cargo from degradative enzymes and the acidity of the stomach. The hydrogel's mucoadhesive properties allow delivery and increased penetration of the cargo to and through the walls of the small intestine. In one embodiment, the hydrogels are made from PEG chains grafted on a poly(methacrylic acid) (PMAA) backbone, hereinafter referred to as P(MAA-g-EG). [Nakamura, K.; Murray, R. J.; Joseph, J. I.; Peppas, N. A.; Morishita, M.; Lowman, A. M. “Oral Insulin Delivery Using P(MAA-g-EG) Hydrogels: Effects of Network Morphology on Insulin Delivery Characteristics” J. Control. Release 2004, 95, 589-599, hereinafter “Nakamura et al.”] The pH-responsive properties of this hydrogel allow the gel to contract in the acidic conditions of the stomach, protecting its contents, and expand in the basic environment of the small intestine to release the contents. This is accomplished via interpolymer complexes forming (stomach) and dissociating (intestine) as a result of temporal hydrogen bonding between the carboxylic acid protons of the backbone and the ether oxygen atoms of the PEG chains. [Nakamura et al.]

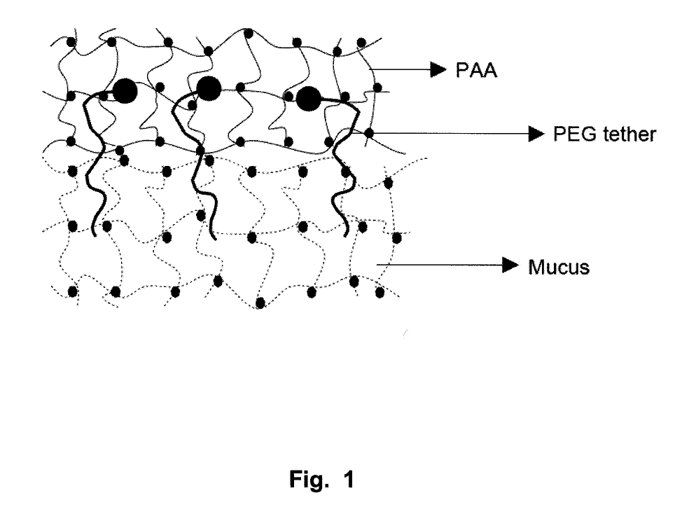

Additionally, acrylic-based polymers have been shown to be mucoadhesive, [Park, H.; Robinson, J. R. “Mechanisms of Mucoadhesion of Poly(acrylic acid) Hydrogels” Pharm. Res. 1987, 4, 457-464.] and PEG grafts increase mucoadhesion by allowing the interpenetration of the carrier through the mucus by an entanglement interaction with the mucins (glycosylated proteins) as illustrated in FIG. 1. [Serra, L.; Domenech, J.; Peppas, N. A. “Design of Poly(ethylene glycol)-Tethered Copolymers as Novel Mucoadhesive Drug Delivery Systems” Eur. J. Pharm. Biopharm. 2000, 50, 27-46.]

In addition, PEG chains of the hydrogel may be grafted to wheat germ agglutinin (WGA), a lectin, to improve residence time and absorption of the drug. [Wirth, M.; Gerhardt, K.; Wumm, C.; Gabor, F. “Lectin-Mediated Drug Delivery: Influence of Mucin on Cytoadhesion of Plant Lectins in Vitro” J. Control. Release 2002, 79, 183-191.] WGA increases mucoadhesion through the specific binding of WGA with the dangling carbohydrate portions of the mucins of the mucosal lining. Carbon nanotubes may be loaded into the hydrogel [Nakamura et al.] and carried through the gastrointestinal tract into the small intestine for direct delivery of the mitigating SWCNTs into the intestinal crypt cells. Since the mucosal layer of one exposed to radiation is likely to be compromised, permeation through the mucosal layer for this purpose should be relatively easier.

The carbon nanotubes contemplated herein for radiation treatment can be made by any known technique (e.g., arc method, laser oven, chemical vapor deposition, flames, HiPco, etc.) and can be in a variety of forms, e.g., soot, powder, fibers, “bucky papers,” etc. Such carbon nanotubes include, but are not limited to, single-wall carbon nanotubes (SWNTs), multi-wall carbon nanotubes (MWNTs), double-wall carbon nanotubes (DWNTs), buckytubes, fullerene tubes, carbon fibrils, carbon nanotubules, stacked cones, horns, carbon nanofibers, vapor-grown carbon fibers, and combinations thereof. In particular embodiments, such carbon nanotubes are generally selected from single-wall carbon nanotubes, double-wall carbon nanotubes, multi-wall carbon nanotubes, small diameter carbon nanotubes, and combinations thereof. In some embodiments, the carbon nanotubes may be predominantly single-wall carbon nanotubes, while in other embodiments the carbon nanotubes may be predominantly double-wall carbon nanotubes. In yet other embodiments, the carbon nanotubes may be predominantly multi-wall carbon nanotubes.

The carbon nanotubes may comprise a variety of lengths, diameters, chiralities (helicities), number of walls, and they may be either open or capped at their ends. Furthermore, they may be chemically functionalized in a variety of manners. In particular, functionalization to confer water solubility is generally desirable. The carbon nanotubes may include semiconducting (bandgaps ˜1-2 eV), semi-metallic (bandgaps ˜0.001-0.01 eV) or metallic carbon nanotubes (bandgaps ˜0 eV), and more particularly mixtures of the three types.

Chemically functionalized carbon nanotubes, as used herein, comprise the chemical modification of any of the above-described carbon nanotubes. Such modifications can involve the nanotube ends, sidewalls, or both. Chemical modification, according to the present invention, includes, but is not limited to, covalent bonding, ionic bonding, chemisorption, intercalation, surfactant interactions, polymer wrapping, cutting, solvation, and combinations thereof. For some exemplary kinds of chemical modifications, see Liu et al., “Fullerene Pipes,” Science, 280, pp. 1253-1256 (1998); Chen et al., “Solution Properties of Single-Walled Carbon nanotubes,” Science, 282, pp. 95-98 (1998); Khabashesku et al., “Fluorination of Single-Wall Carbon Nanotubes and Subsequent Derivatization Reactions,” Acc. Chem. Res., 35, pp. 1087-1095 (2002); Sun et al., “Functionalized Carbon Nanotubes: Properties and Applications,” Acc. Chem. Res., 35, pp. 1096-1104 (2002); Holzinger et al., “Sidewall Functionalization of Carbon Nanotubes,” Angew. Chem. Int. Ed., 40(21), pp. 4002-4005 (2001); Bahr et al., “Covalent chemistry of single-wall carbon nanotubes,” J. Mater. Chem., 12, pp. 1952-1958 (2002); Gu et al., “Cutting Single-Wall Carbon Nanotubes through Fluorination,” Nano Letters, 2(9), pp. 1009-1013 (2002), O′Connell et al., “Reversible water-solubilization of single-walled carbon nanotubes by polymer wrapping,” Chem. Phys. Lett., 342, pp. 265-271 (2001), Dyke et al., “Solvent-Free Functionalization of Carbon Nanotubes,” J. Am. Chem. Soc., 125, pp. 1156-1157 (2003), Dyke et al., “Unbundled and Highly Functionalized Carbon Nanotubes from Aqueous Reactions,” Nano Lett., 3, pp. 1215-1218 (2003).

Carbon nanotubes can also be physically modified by techniques including, but not limited to, physisorption, plasma treatment, radiation treatment, heat treatment, pressure treatment, and combinations thereof, prior to being treated according to the methods of the present invention. In some embodiments of the present invention, carbon nanotubes have been both chemically and physically modified.

Any particular carbon nanotube type may be used in purified form or in raw form from the synthetic process. Carbon nanotubes can be in their raw, as-produced form, or they can be purified by a purification technique. Furthermore, mixtures of raw and purified carbon nanotubes may be used. For some exemplary methods of carbon nanotube purification, see Rinzler et al., “Large-Scale Purification of Single-Walled Carbon Nanotubes: Process, Product, and Characterization,” Appl. Phys. A, 67, pp. 29-37 (1998); Zimmerman et al., “Gas-Phase Purification of Single-Wall Carbon Nanotubes,” Chem. Mater., 12(5), pp. 1361-1366 (2000); Chiang et al., “Purification and Characterization of Single-Wall Carbon nanotubes,” J. Phys. Chem. B, 105, pp. 1157-1161 (2001); Chiang et al., “Purification and Characterization of Single-Wall Carbon Nanotubes (SWNTs) Obtained from the Gas-Phase Decomposition of CO (HiPco Process),” J. Phys. Chem. B, 105, pp. 8297-8301 (2001).

In some embodiments, the carbon nanotubes may be separated on the basis of a property such as length, diameter, chirality, electrical conductivity, number of walls, and combinations thereof, prior to being treated according to the methods described herein. See Farkas et al., “Length sorting cut single wall carbon nanotubes by high performance liquid chromatography,” Chem. Phys. Lett., 363, pp. 111-116 (2002); Chattopadhyay et al., “A Route for Bulk Separation of Semiconducting from Metallic Single-Wall Carbon nanotubes,” J. Am. Chem. Soc., 125, 3370-3375 (2003); Bachilo et al., “Structure-Assigned Optical Spectra of Single-Walled Carbon Nanotubes,” Science, 298, 2361-2366 (2002); Strano et al., “Electronic Structure Control of Single Walled Carbon Nanotube Functionalization,” Science, 301, pp. 1519-1522 (2003).

Carbon nanotubes useful in the treatment of radiation exposure or radical damaging process may include those functionalized with a radical scavenger. The radical scavenger-carbon nanotube conjugates can be used as a means of radiation protection as described hereinabove. Radical scavengers may include, for example phenols. Butylated hydroxyanisole (BHA) and butylated hydroxytoluene (BHT) are well known food preservatives that are excellent radical scavengers. In some embodiments, it is shown that radical scavenger-nanostructured conjugates that include these compounds, among others, attached to SWNTs, for example, serve as effective radical traps. 4-(2-Aminoethyl)-2,6-bis(1,1-dimethylethyl)phenol (amino-BHT, compound 3, see Scheme 1 in Examples below) groups are associated with nano-engineered materials. The amino-BHT groups can be associated with SWNTs that have carboxylic acid groups via acid-base association or via covalent attachment. The PEGylated carbon nanotubes can also sequester desired molecules, for example Misoprostol. Furthermore, the SWNTs could also have poly(ethylene glycol) (PEG) chains associated with them to enhance the solubility of the nano-engineered materials in water and buffered systems. Likewise 4-(2-carboxyethyl)-2,6-bis(1,1-dimethylethyl)phenol (carboxy-BHT, compound 4, Scheme 2) could be associated with aminated SWNTs (i.e. SWNTs that are carboxylated, then aminated via interaction with poly(ethylene imine, for example), again via acid base association. In some embodiments, the present invention provides a means of attachment of 2,6-di(tert-butyl)phenols (BHT and BHA analogues) to SWNTs, and use of these conjugates as delivery agents to quench large amounts of radicals that may be established in a cell due to oxidative stress or radiation-induced pathways.

Many other radical scavengers may be appended to the sidewalls of water soluble SWNTs via acid-base (shown below), covalent (shown below), or non-covalent (pi-pi interactions or Van der Waals interactions, not shown) functionalization protocols. In some embodiments, the parent PLURONIC®-wrapped SWNT can show efficacy in radical quenching as well. Shown below are a series of compounds that could be used including 3, 4, 5, and 6 as well as known therapeutic radical scavengers such as, Lavendustin B and Amifostine, to name just two. One skilled in the art will recognize that several other means of derivatizing and attaching radical scavengers to SWNTs or DWNTs or MWNTs may be possible. Other radical scavengers useful in practicing the method of treatment contemplated herein include thiols, such as glutathione, and polythiols such as poly(mercaptopropyl)methylsiloxane.

As mentioned above, it is generally desirable to provide carbon nanotubes that possess a degree of water solubility for administration. In particular carbon nanotubes conjugated to PEG polymer systems should provide a biocompatible water soluble system. Applicants expect that the PEG-conjugate will also allow an exogenous radical scavenger to be administered.

In general, the present disclosure provides a composition that includes a carbon nanotube as described above. The carbon nanotube may be rendered substantially water soluble and a radical trapping agent is associated with the carbon nanotube forming a radical scavenger-nanotube conjugate. As previously described the radical trapping agents include phenols and thiols. The radical trapping agent may be at least one selected from the group consisting of compounds 3, 4, 5, 6, Amifostine, and Lavendustin B, 13, 16 as shown below.

The radical trapping agent may be associated with the carbon nanotube through an ionic acid-base interaction, a covalent bond, a pi-pi interaction, a Van der Waals interaction, sequestration, and physisorption. Acid-base interactions are readily accessible via cut nanotubes or at sidewall defects that display carboxylic acid functionality, for example. Covalent functionalization can be accessed by diazonium decomposition chemistry described in co-pending application Ser. No. 10/632,419 which is incorporated by reference herein in its entirety. Moreover, the sidewall of the carbon nanotube itself is an excellent radical scavenger, as shown here, and could be used in its poly-wrapped form so as to confer it with water-solubility.

### EXAMPLES

The following experimental examples are included to demonstrate particular aspects of the present invention. It should be appreciated by those of skill in the art that the methods disclosed in the examples that follows merely represent exemplary embodiments of the present invention. However, those of skill in the art should, in light of the present disclosure, appreciate that many changes can be made in the specific embodiments described and still obtain a like or similar result without departing from the spirit and scope of the present invention.

### Example 1

### a) Synthesis of 3

This follows, in part, a known protocol. (The conversion of nitrile 2 to an acid should be possible via this scheme as well).

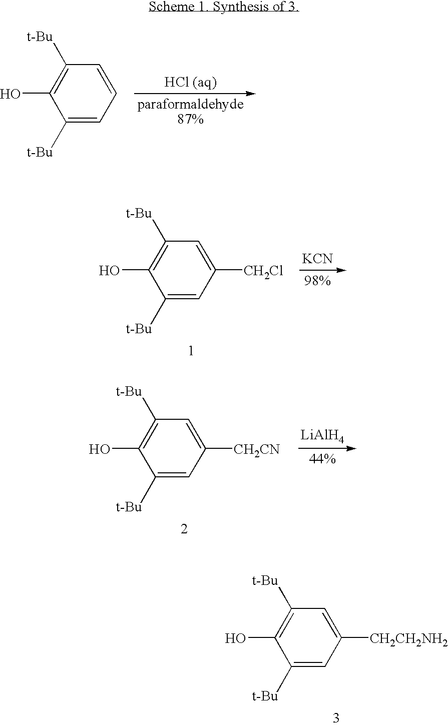

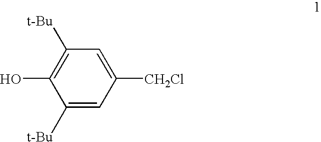

2,6-Di-tert-butyl-4-chlorophenol (1). A 100 mL round bottom flask equipped with a magnetic stir bar was charged with commercially available 2,6-di-tert-butylphenol (5.0 g, 24 mmol), paraformaldehyde (15 g, 0.5 mol), and concentrated hydrochloric acid (45 mL). The mixture was stirred vigorously at 85° C. for 1 h under a nitrogen atmosphere. The reaction flask was allowed to cool to room temperature and the organic layer was then collected. The aqueous layer was then extracted with hexanes. The organic layers were combined and washed with water until the pH was neutral. The organic layer was dried with MgSO₄, filtered and the solvent was removed under reduced pressure. The resulting yellow oil was used without further purification (87%).

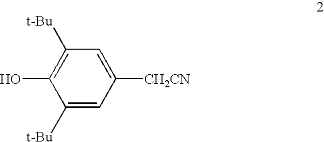

2-(3,5-Di-tert-butyl-4-hydroxyphenyl)acetonitrile (2). A 250 mL round bottom flask equipped with a magnetic stir bar and reflux condenser was charged with KCN (3.15 g, 48 mmol) and aqueous acetonitrile (4:1 acetonitrile:water, 125 mL) and stirred until dissolved. Compound 1 (˜5 g, ˜20 mmol) was dissolved in acetonitrile and the solution was then added drop wise through the condenser into the stirring cyanide solution. The reaction stirred for 5 min and was then diluted with aqueous hexanes. The mixture was extracted with hexanes. The combined organic layers were washed with water, dried with MgSO₄, filtered and purified using column chromatography (silica gel as stationary phase). The product was further purified via crystallization from aqueous methanol to yield light pink crystals (98%).

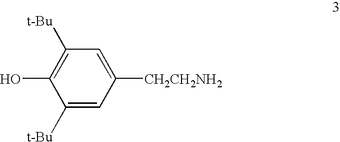

2,6-Di-tert-butyl-4-(2-aminoethyl)phenol (3). [Um, S.; Lee, J.; Kang, Y.; Baek, D. Dyes and Pigments, 2006, 70, 84.] Compound 2 (2.65 g, 10.8 mmol) was dissolved in anhydrous diethyl ether (50 mL). A 250 mL round bottom flask was equipped with a magnetic stir bar was charged with LiAlH₄ (1.05 g, 27.7 mmol) and anhydrous ethyl ether (40 mL) and cooled to 0° C. The solution of compound 2 was added dropwise. The mixture was vigorously stirred and gently refluxed for 3 h under a nitrogen atmosphere and then cooled to 0° C. NaOH (3 M) was added to decompose any excess LiAlH₄. The mixture was then filtered and extracted with diethyl ether. The organic layers were combined, washed with water, dried over MgSO₄, and filtered. The solvent was removed under reduced pressure. The product was purified by recrystallization from hexanes to yield light orange crystals (44%). ¹H NMR (400 MHz, CDCl₃, ppm): 6.99 (s, 2H); 5.08 (broad s, 1H); 2.93 (t, J=7.2 Hz, 2H); 2.66 (t, J=7.2 Hz, 2H), 1.42 (s, 18H).

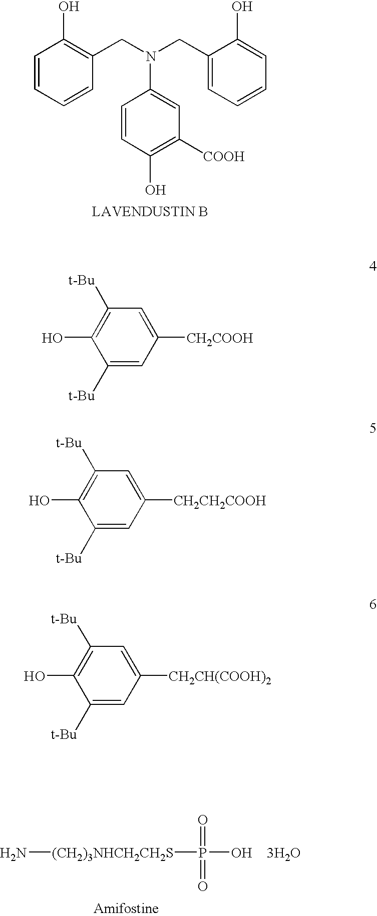

As shown in Scheme 2 above, several other radical scavenging molecules may be constructed de novo, or are commercially available and amenable for attachment to nanostructured materials such as SWNTs.

### b) Synthesis of 11

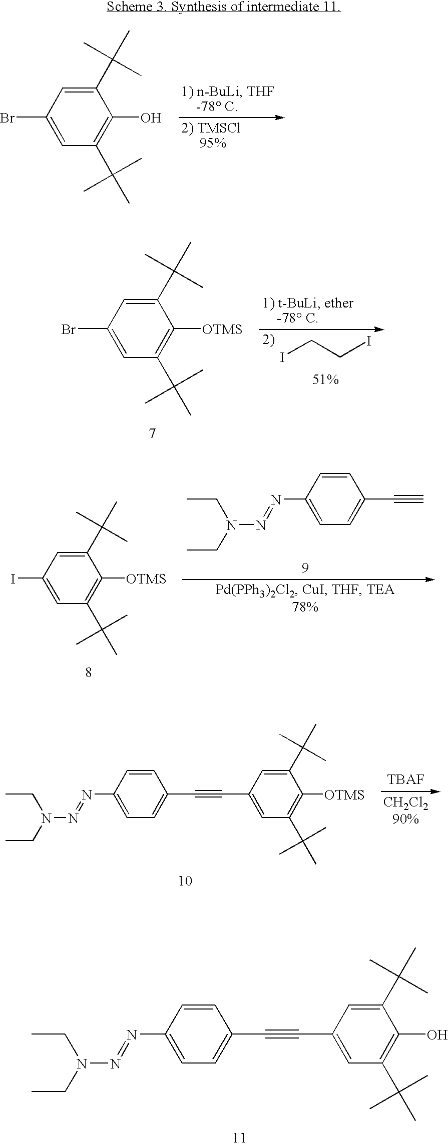

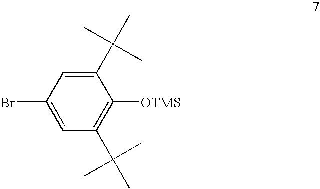

(2,6-Di-tert-butyl-4-bromophenoxy)trimethylsilane (7). A oven dried 100 mL round bottom flask equipped with a stir bar was charged with commercially available 2,6-di-tert-butyl-4-bromophenol (2.85 g, 10 mmol), (trimethylsilyl)methyl chloride (1.84 g, 15 mmol) and THF (50 mL) and then cooled to −78° C. N-butyllithium (0.96 g, 15 mmol) was slowly added and the mixture was stirred for 30 min. The mixture was allowed to come to room temperature and then poured into water. The product was extracted with hexanes and the combined organic layers were washed with water. The organic layer was dried with MgSO₄, filtered, and the solvent was removed under reduced pressure. The product was purified by column chromatography (silica gel, hexanes as eluent) to provide 1.91 g of title product 7 (95%). ¹H NMR (400 MHz, CDCl₃, ppm) 7.32 (s, 2H), 1.38 (s, 2H), 0.38 (s, 9H).

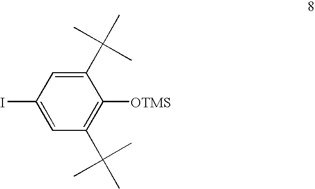

(2,6-Di-tert-butyl-4-iodophenoxy)trimethylsilane (8). An oven dried 100 mL round bottom flask equipped with a stir bar was charged with compound 7 (3.40 g, 9.5 mmol) and ether. The mixture was cooled at −78° C. and tert-butyllithium (1.83 g, 28.5 mmol) was slowly added. The resulting solution was stirred for 1 h and 1,2 diiodoethane (5.35 g, 19 mmol) was added. The mixture was stirred at −78° C. for 1 h and then allowed to come to room temperature. The solution was poured into water and extracted with hexanes. The combined organic layers were washed with water and dried with MgSO₄. The product was filtered and the solvent removed under reduced pressure. The resulting material (1.47 g) was a mixture of 7 and the desired product 8 (51%). ¹H NMR (400 MHz, CDCl₃, ppm) 7.34 (s, 2H), 1.38 (s, 2H), 0.38 (s, 9H).

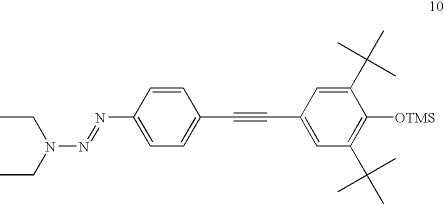

Compound 10. An oven dried 100 mL round bottom flask equipped with a stir bar was charged with compound 8 (1.47 g of the mixture), 1-acetylenephenyl(3,3-diethyl)triazene 9 [Li, G.; Wang, X.; Wang, F. Tetrahedron Lett. 2005, 46, 8971-8973] (0.40 g, 2 mmol), PdCl₂(PPh₃)₂ (0.042 g, 0.6 mmol), CuI (0.025 g, 1.3 mmol), triethylamine (2 mL) and well degassed THF (30 mL) were stirred at 60° C. for a number of h until analysis showed conversion of 5. The mixture was filtered and poured into saturated NH₄Cl and extracted with dichloromethane. The combined organic layers were washed with water and dried with MgSO₄. The product was filtered and the solvent removed under reduced pressure. The product was purified by column chromatography (silica gel as stationary phase, 1:3 dichloromethane to hexanes) to yield 0.83 g (78%) of the desired product 10. ¹H NMR (400 MHz, CDCl₃, ppm) 7.41 (d, J=8.4, 2H), 7.34 (s, 2H), 7.29 (d, J=8.4, 2H), 7.36 (s, 2H), 3.78 (q, J=14.3, 4H), 1.45 (s, 16H), 1.27 (t, J=14.3, 6H), 0.38 (s, 9H).

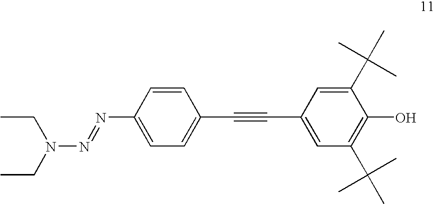

Compound 11. To a 100 mL round bottom flask equipped with a magnetic stir bar, compound 10 dissolved in dichloromethane (30 mL) and tetra-n-butylammonium fluoride (3 mL, 3 mmol, 1.0 M in THF) were stirred overnight at room temperature. The color changed from red to green. The product was isolated by filtering the solution through a silica gel plug and washing with 1:1 dichloromethane and hexane to give an orange solution. The solvent was removed under reduced pressure to provide the red solid (0.48 g, 90%). ¹H NMR (400 MHz, CDCl₃, ppm) 7.49 (d, J=8.4, 2H), 7.38 (d, J=8.4, 2H), 7.36 (s, 2H), 3.78 (q, J=14.3, 4H), 1.45 (s, 16H), 1.27 (t, J=14.3, 6H). ¹³C NMR (125 MHz, CDCl₃, ppm) 154.2, 150.6, 136.1, 132.1, 128.6, 120.3, 114.4, 90.2, 34.4, 30.2, 30.0. m.p. 61-63° C.

### c) Synthesis of 13

The following functionalizations were completed. The source of all SWNTs was the HiPco SWNT reactor from Rice University. The PLURONIC® used was F108. The SWNTs for the parent pluronic-wrapped tubes 12 and for the starting of 13 were prepared according to reference 2 as decants using PLURONIC® F108 as the surfactant. The SWNTs for 16 were prepared and cut at room temperature using oleum and nitric acid according to Chen, Z.; Kobashi, K.; Rauwald, U.; Booker, R.; Fan, H.; Hwang, W. F.; Tour, J. M. J. Am. Chem. Soc. 2006, 32, 10568.

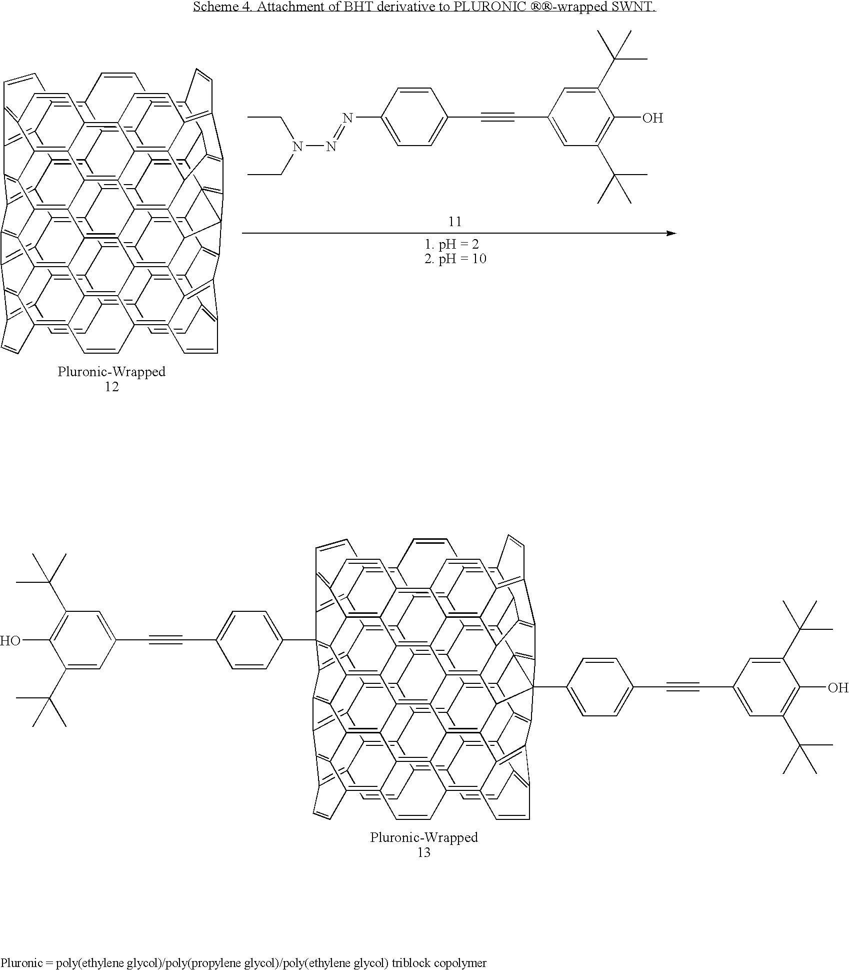

### d) Synthesis of 14-16

Synthesis of covalently appended BHT derivatized SWNTs (13). The pH of PLURONIC® (1 wt % in water) wrapped SWNTs 12 (50 mL) [Moore, V. C.; Strano, M. S.; Haroz, E.; Hauge, R. H.; Smalley, R. E. Nano Lett. 2003, 10, 1379.] was adjusted with enough concentrated HCl to lower the pH to 2. Compound 11 (25 mg, 0.62 mmol) was dissolved in acetonitrile (2 mL) and then added to the SWNT solution. [Hudson, J. H.; Jian, H.; Leonard, A. D.; Stephenson, J. J.; Tour, J. M. Chem. Mater. 2006, 18, 2766.] The mixture was stirred for 20 min and the pH was adjusted to 10 by adding NaOH (40%) dropwise. The mixture was then dialyzed (dialysis bag MWCO 50 K) in PLURONIC® (1 wt % in water) for 5 days to purify the material and afford 13.

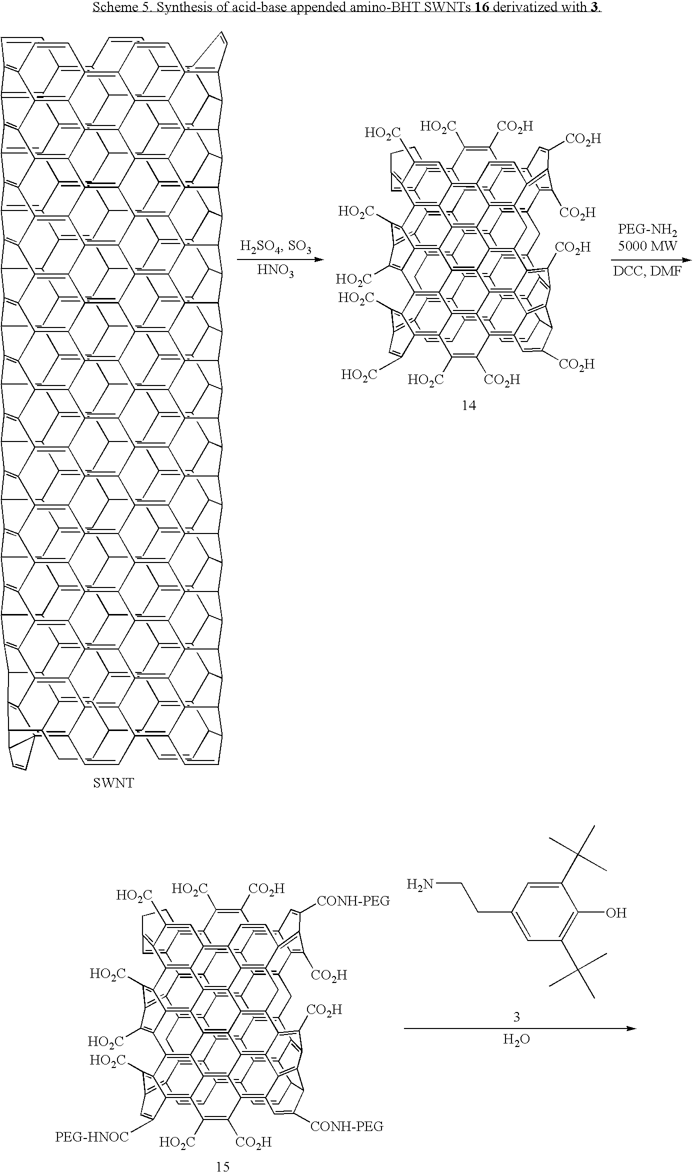

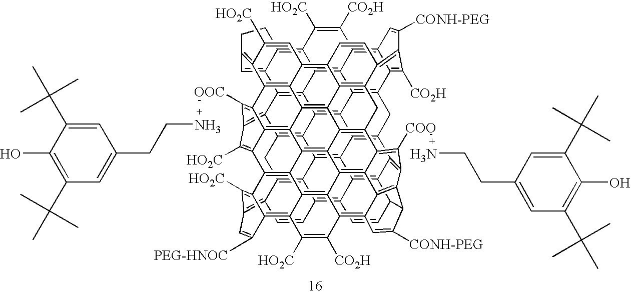

Cut Single Walled Carbon Nanotubes (14). [Chen, Z.; Kobashi, K.; Rauwald, U.; Booker, R.; Fan, H.; Hwang, W. F.; Tour, J. M. J. Am. Chem. Soc. 2006, 32, 10568.] Purified SWNTs (100 mg, 8.3 mmol) and oleum (50 mL) were added to a 300 mL Erlenmeyer flask equipped with a stir bar and stirred overnight under a nitrogen atmosphere. Nitric acid (34 mL, 70%) was poured into a 100 mL graduated cylinder. Oleum (50 mL) was then CAREFULLY added to the nitric acid and then immediately poured into the suspension of SWNTs. The mixture was stirred for 2 h at room temperature and then quenched over 500 g of ice. The mixture was filtered on a polycarbonate membrane (0.22 μm). To neutralize the moist material, it was then resuspended in a minimal amount of methanol and then ethyl ether (300 mL) was added to flock the SWNTs. The neutralization step was repeated until the pH of the ethyl ether was neutral.

PEGylation of the SWNTs (15). An oven dried 100 mL round bottom flask equipped with a stir bar was charged with 14 (0.063 g, 5.2 mmol) and anhydrous DMF (50 mL). The mixture was vigorously stirred for 15 min under a nitrogen atmosphere. N,N′-dicyclohexylcarbodiimide (DCC, 1.08 g, 5.2 mmol) was added followed by poly(ethylene glycol) (0.50 g, 0.1 mmol Mw 5000). The mixture was stirred overnight and purified by dialysis (MWCO 50K) for 5 d. The solution of product 15 was filtered through glass wool and was used without further purification.

Acid-base appended amino-BHT derivatized SWNTs (16). Compound 15 (0.0006 g, 0.05 mmol) was added to a 50 mL round bottom flask equipped with a stir bar. Amino-BHT 3 (0.012 g, 0.05 mmol) was dissolved in DMF (1 mL) and added to the mixture to stir overnight. The material was purified by dialysis (MWCO 50K).

### e) Synthesis of 17

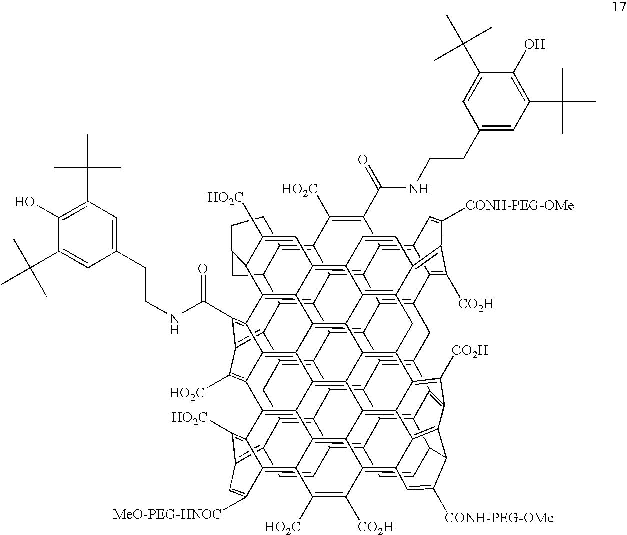

Covalently bound amine-BHT derivatized PEGylated US-SWCNTs (17). DCC (0.026 g, 0.126 mmol) was quickly added to a stirring solution of PEGylated US-SWCNT 15 (0.003 g, 0.25 mmol), under a nitrogen atmosphere in anhydrous DMF. After 10 min, 2,6-di-tert-butyl-4-(2-aminoethyl)phenol 3 (0.016 g, 0.064 mmol) was added quickly, in the same fashion as DCC. The reaction was left stirring overnight at room temperature. The mixture was purified in the same way as the PEGylated US-SWCNT solution 15.

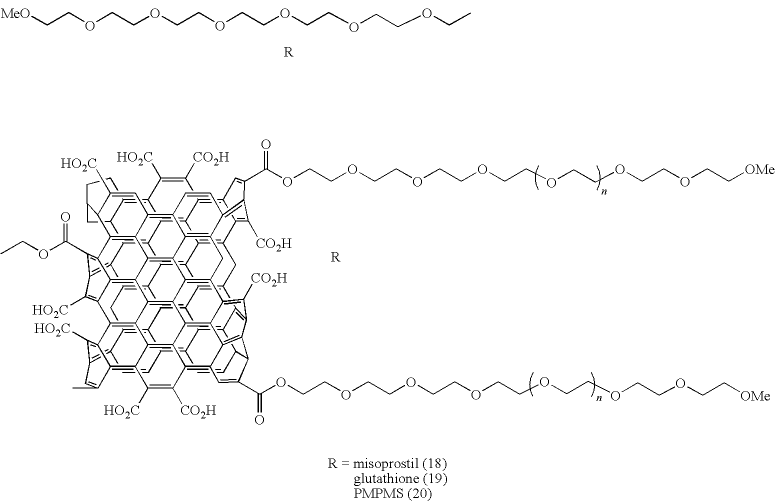

Misoprostol PEGylated SWNTs (18). PEGylated SWNTs 15 (4 mL, 61 mg/mL) were added to a 5 mL glass vial equipped with a stir bar. Misoprostol (0.6 mg, 1.6×10⁻³ mmol) was dissolved in methanol (0.5 mL) and added into the stirring mixture. The solution was allowed to stir for 10 min, then sonicated in a bath sonicator for an additional 10 min, to ensure full sequestration. The volume of the solution was reduced under vacuum until it had decreased by twice the volume of methanol added. Deionized water was added to the solution to bring it back to the original volume of the original PEGylated SWNT solution. The contents were sonicated again for 10 minutes.

Glutathione PEGylated SWNTs (19). PEGylated SWNTs 15 (0.05 mg/mL) were added to a 5 mL glass vial equipped with a stir bar. Glutathione (1 mg, 3.25×10⁻³ mmol) was added to the stirring mixture. The solution was allowed to stir for 10 min, then sonicated in a bath sonicator for an additional 10 min, to ensure full sequestration.

PMPMS PEGylated SWNTs (20). PEGylated SWNTs 15 (5 mL, 69.5 mg/L) were added to a 10 mL glass vial equipped with a stir bar. PMPMS (poly(mercaptopropyl)methylsiloxane (5500 MW, 55 mg) was dissolved in tetrahydrofuran (THF, 0.96 mL) and added into the stirring mixture. The solution was allowed to stir for 10 min, then sonicated in a bath sonicator for an additional 10 min, to ensure full sequestration. The volume of the solution was reduced under vacuum until it had decreased by twice the volume of THF added. Deionized water was added to the solution to bring the solution back to the original volume of the original PEGylated SWNT solution. The contents were sonicated again for 10 minutes.

### Example 2

### Oxygen Radical Absorbance Capacity (ORAC) Assay

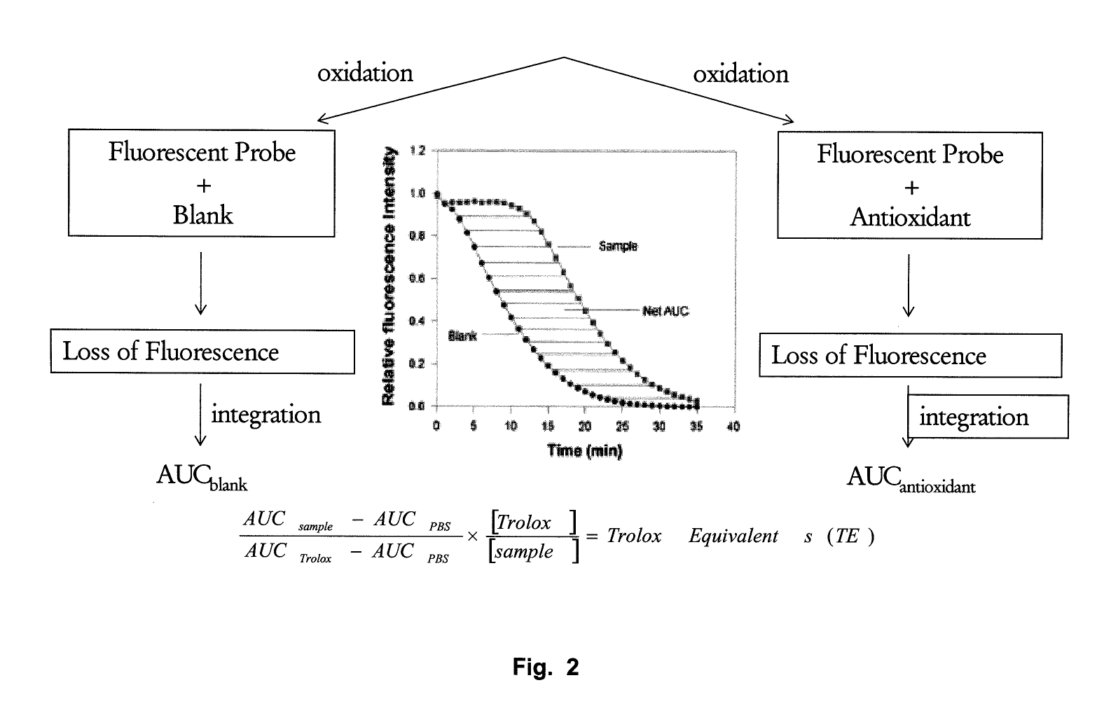

The oxygen radical absorbance capacity assay measures the oxidative degradation of the fluorescent molecule after being mixed with free radical generators (such as azo-initiator compounds). Azo-initiators are considered to produce peroxyl free radical by heating, which damages the fluorescent molecule, resulting in the loss of fluorescence. Antioxidant is able to protect the fluorescent molecule from the oxidative degeneration. The degree of protection is quantified using a fluorometer. The fluorescent intensity decreases as the oxidative degeneration proceeds, and this intensity is recorded for typically 35 minutes after the addition of the free radical generator (azo-initiator). The degeneration (or decomposition) of fluorescein that is measured as the fluorescence delay becomes less prominent by the presence of antioxidants. Decay curves (fluorescence intensity vs. time) are recorded and the area between two decay curves (with or without antioxidant) is calculated. Subsequently, the degree of antioxidant-mediated protection is quantified using the antioxidant (TROLOX®, a vitamin E analogue) as a standard. Different concentrations of TROLOX® are used to make a standard curve, and test samples are compared to this. Results for test samples are published as “TROLOX® equivalents” or TE (FIG. 2).

All solutions were prepared daily in 75 mM phosphate buffered saline (PBS) at pH 7.4. Fluorescein sodium salt (FL) was prepared at 0.2 μM from a 4 mM stock solution (prepared fresh monthly and stored in the dark at 4° C.). α,α′-Axodiisobutyramidine dihydrochloride (AAPH) was prepared at 0.15 M and kept in an ice bath until added to the system. (+−)-6-Hydroxy-2,5,7,8-tetramethylchromane-2-carboxylic acid (TROLOX®) was prepared at 400 μM.

The experiments were performed in a black sided, clear bottom 96-well plate. In order to account for the background and any fluorescence loss during the overnight experiments, PBS was substituted for AAPH and FL in two wells. Therefore, each sample was analyzed in three wells as follows: (1) Assay=120 μL FL+20 μL sample+60 μL AAPH, (2) Control 1 (minus AAPH)=120 μL FL+20 μL sample+60 μL PBS, (3) Control 2 (minus FL)=120 μL PBS+20 μL sample+60 μl AAPH. The FL, sample, and PBS were added in the appropriate wells. Each experimental run included TROLOX® and PBS as standards. The plate was then incubated at 37° C. for 15 minutes in a Safire2 plate reader (Tecan Systems Inc). Then ice cold 4 mM AAPH was added to the appropriate wells. The fluorescent intensity at 530 nm with 485 nm excitation was monitored every minute for 6 hours.

The background spectrum (control 2) was subtracted from the assay and control 1 results. The assay well results were divided by the control 1 results. The area under the curve (AUC) for the resultant values was computed. The TROLOX® equivalent values were calculated using the equation below. For molar TROLOX® equivalents, concentration was expressed in molarity.

$$\frac{{AUC}_{sample} - {AUC}_{PBS}}{{AUC}_{Trolox} - {AUC}_{PBS}} \times \frac{[ Trolox ]}{[ sample ]} = Trolox Equivalents ( TE )$$

Each sample was run a total of nine times with the above treatment. Averages and standard deviations were calculated.

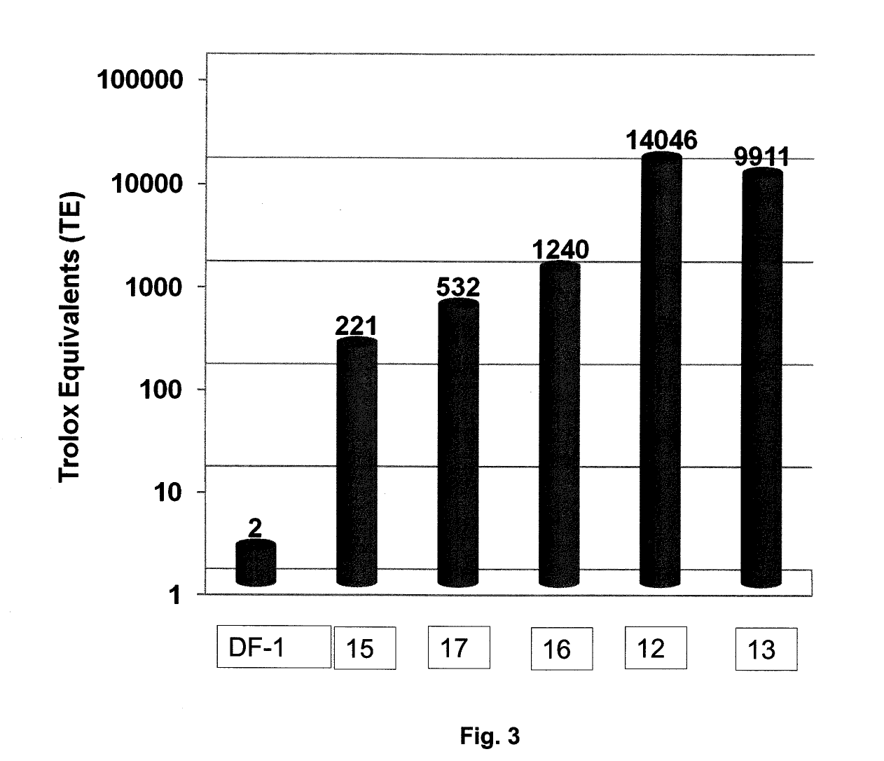

The results of this assay are depicted in FIG. 3. Compound 12 (PLURONIC®© wrapped SWCNTs) gave the highest ORAC number (14046 TE). Once BHT groups were added to the sidewalls of the PLURONIC® wrapped SWCNTs (compound 13), the ORAC number decreased (9911). In contrast, the PEGylated US-SWCNTs (compound 15) produced the lowest value for the ORAC assay (221) with increasing values proportional to BHT added in compounds 17 and 16 (532, 1250). DF-1 is a known C60 radical scavenging derivative as a point of comparison with a value of 2; note the substantial increase in changing from the C60 structure to the carbon nanotube.

### Example 3

### Assessing the Radical Scavenging Potency In Vitro

Compound 16 and compound 17 were tested to prove if the material had radiation protection or mitigation properties using rat small intestine crypt cells (ATCC cat #CRL-1592) as an in vitro assay. A solution of either compound 16 or compound 17 was added to rat small intestine crypt cells grown in medium prior to (protection) and after (mitigation) radiation exposure. When given prior to radiation, the compound solution was added to the cell's medium 2 hours prior to radiation and then removed and replaced with the standard medium solution just before radiation for the protection assay. The cells were exposed to a total of 5 Gy of gamma-radiation with a Cs137 source from a Gamma cell 40 “Exactor” by MDS Nordion at dose rate of 1.10 Gy/minute. In the mitigation test, the compound solution was added to the cell's medium 2 hours after radiation and allowed to incubate for an additional 2 hours (37° C. in 5% CO₂). The cells, thus treated, were removed from their plates with trypsin 48 hours after radiation and the viable cells were counted using a trypan blue permeability assay.

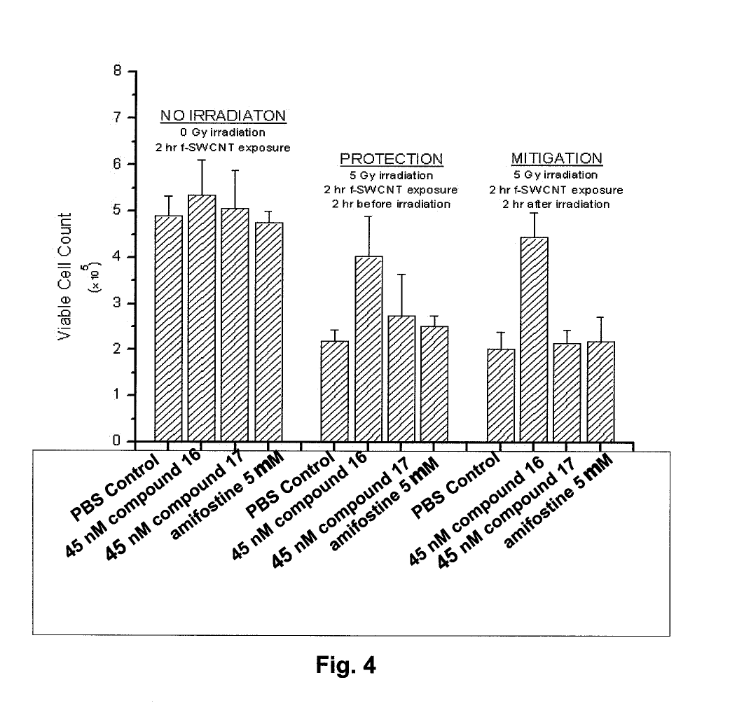

The controls for the irradiation study were a blank phosphate buffered saline (PBS) and medium charged with Amifostine. Amifostine is only active in vivo and was not expected to display significant protection or mitigation properties. Another control of cells not exposed to radiation was run for comparison against the irradiated cells. The cells exposed to compound 16 had a significantly higher rate of survival in both protection and mitigation tests when compared to the controls (FIG. 4).

For the protection assay, viable cell count was observed to be higher for cells exposed to radiation following treatment with compound 16 or compound 17, as compared to blanks or cells treated with Amifostine prior to radiation exposure.

Trypan blue permeability assay—Cytotoxicity of SWCNT Formulations. Human renal epithelial (HRE) and HepG2 liver cells were utilized to assay acute cytotoxicity induced by all BHT derivatized and non-derivatized SWCNTs. The cells were plated at 1×10⁵ cells/well in a 12-well tissue culture treated plate. The cells were allowed to attach overnight at 37° C. in 5% CO₂. The SWCNT samples were added at a dose concentration of 66 nM (17 mg/L) for pluronic wrapped SWCNTs and 332 nM (83 mg/L) for all PEGylated US-SWCNT samples. Triton-X at 1 wt % in water was utilized as the toxic control. After 24 hours exposure to the SWCNT solutions, the cells were removed from the plate with trypsin. Cell viability was assayed utilizing a Beckman Coulter Vi-Cell XR employing a trypan blue permeability assay. The viable cell counts were normalized to the PBS control. These tests showed that there was little to no toxicity from the nanotube samples.

### Example 4

### Assessing the Radical Scavenging Potency In Vivo

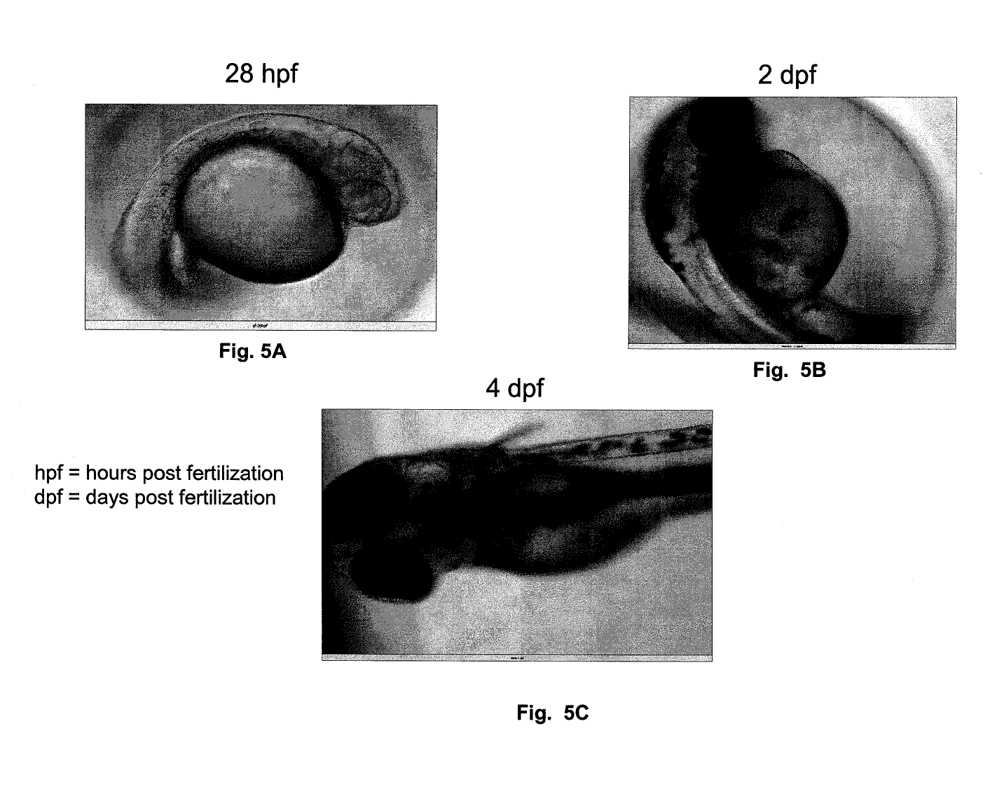

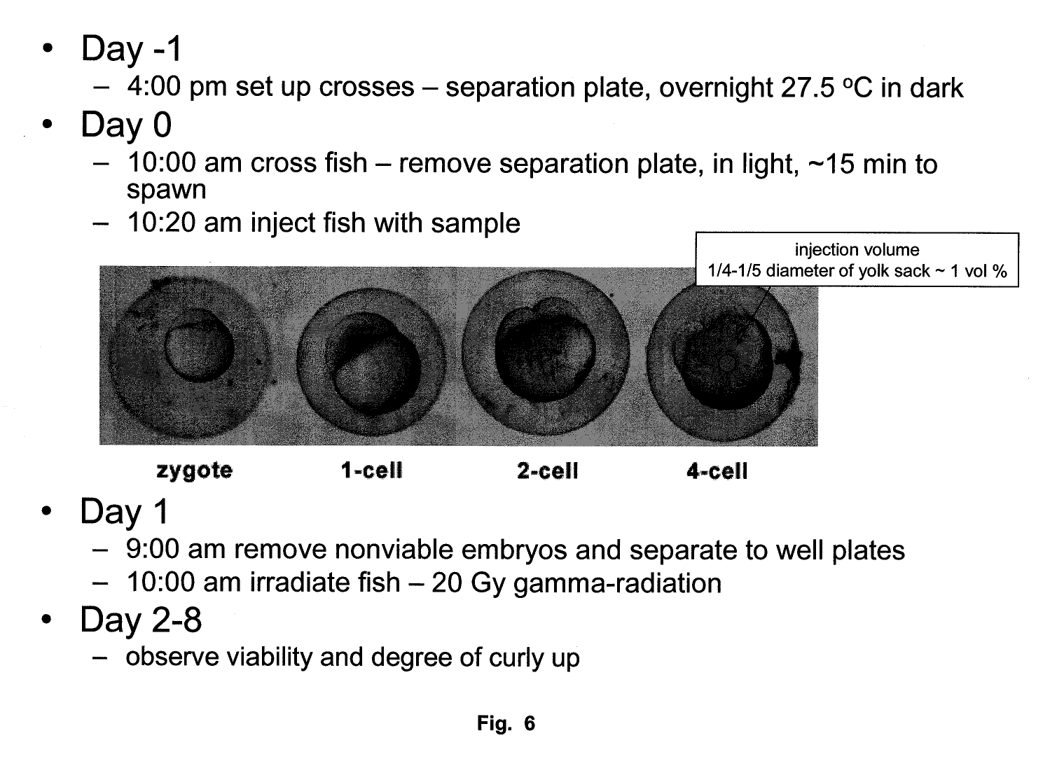

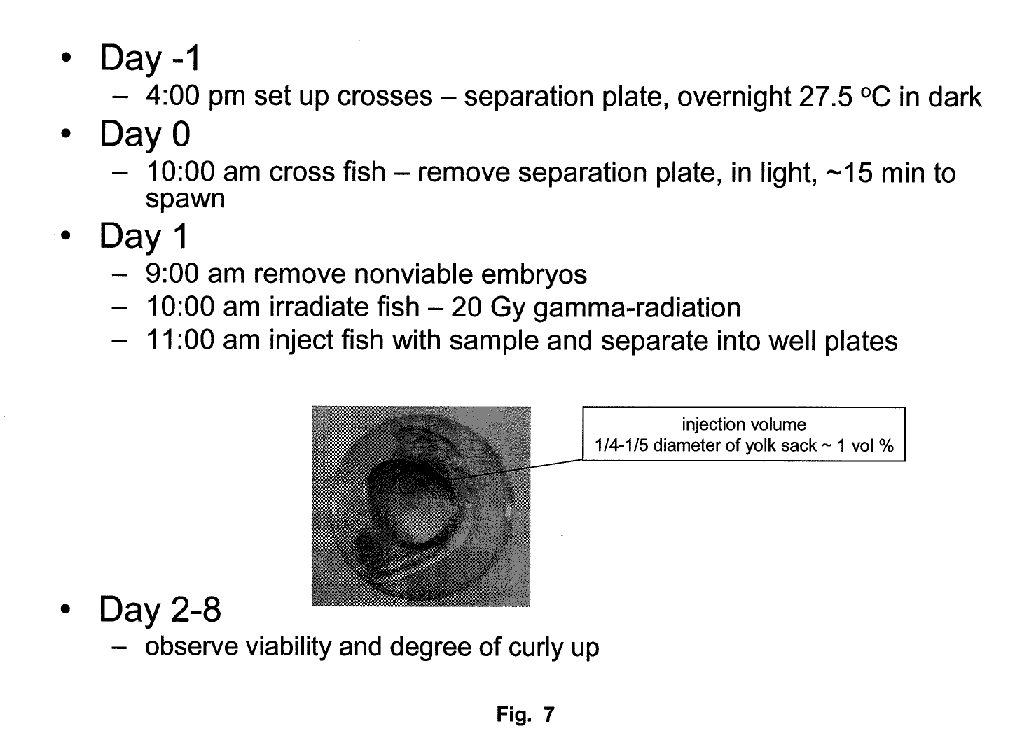

Zebrafish provide an ideal in vivo model for several reasons including, for example, upkeep that is substantially less than required for mice and rats, they represent a vertebrate species for which the entire genome has been sequenced, and large numbers of embryos can be developed synchronously facilitating high throughput screens. Zebrafish have been used to model human responses to radiation. The short maturation time of the embryos from fertilization to hatching, roughly one week, makes them ideal candidates for producing relevant data quickly for an in vivo radiation study (FIGS. 5A-5C). [Kari, G.; Rodeck, U.; Dicker, A. P. “Zebrafish: An emerging model system for human disease and drug discovery,” Nature, 2007, 82(1), 70-80, hereinafter “Kari et al.”] The zebrafish protection assay was done in nine days on 99 or 100 viable embryos (FIG. 6). The first day two adult zebrafish (male and female) were placed in the same tank overnight with a separation plate between them at 27.5° C. in the dark. The following morning the plate was removed, the lights were turned on and the fish were allowed to spawn for 15 minutes. Then, for the protection assay, the resulting fertilized eggs were collected and the carbon nanotube solution was injected into the yolk sac of the embryos. On the third day the embryos were removed and separated into 96-well plates. One hour later the embryos were exposed to 20 Gy of gamma-radiation (FIG. 6). The young zebrafish were observed in days four through nine for viability and the degree of curly up. The mitigation assay was performed in the same manner except the carbon nanotube solution was not injected until one hour after irradiation (FIG. 7). The control set was not exposed to gamma irradiation.

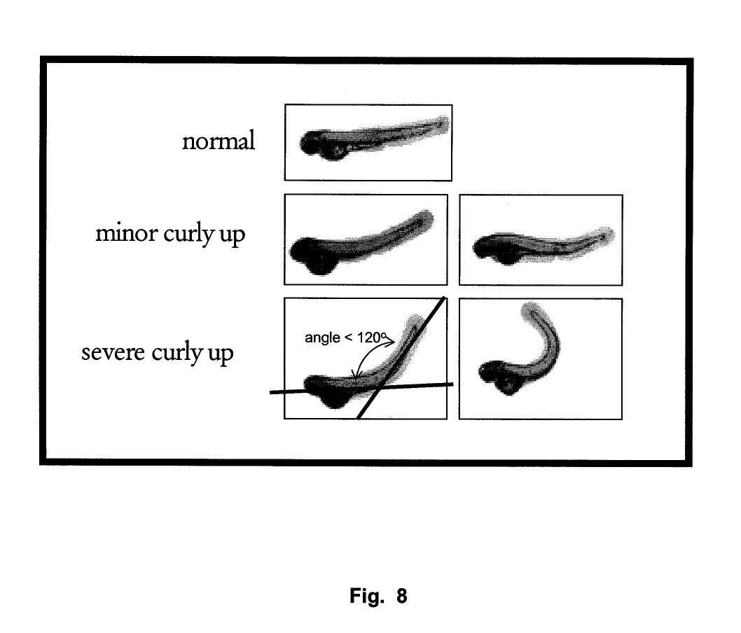

The extent of curly up was assessed according to the quantification of the angle measured between the body and the tail of the fish (FIG. 8). The degree of curly up provides an assessment of radiation-induced damage. [Kari et al.] The minor cases display an angle less than 120°, while a severe case constitutes an angle measurement of greater than 120°. In very severe cases, the complete curling of the tail can be observed after six days of development.

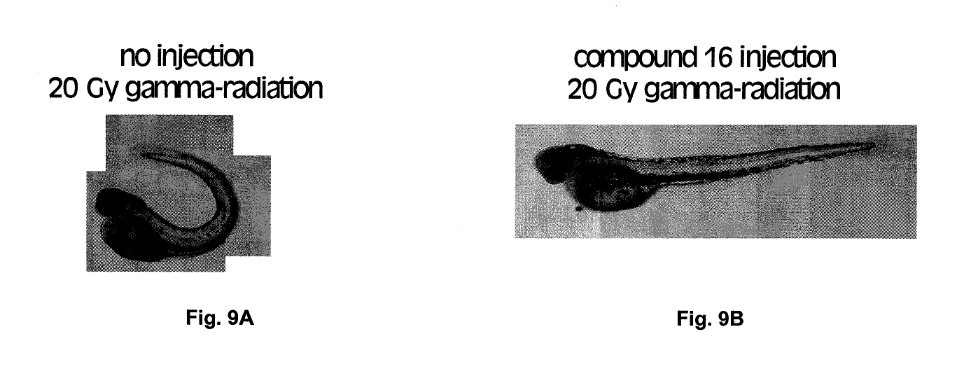

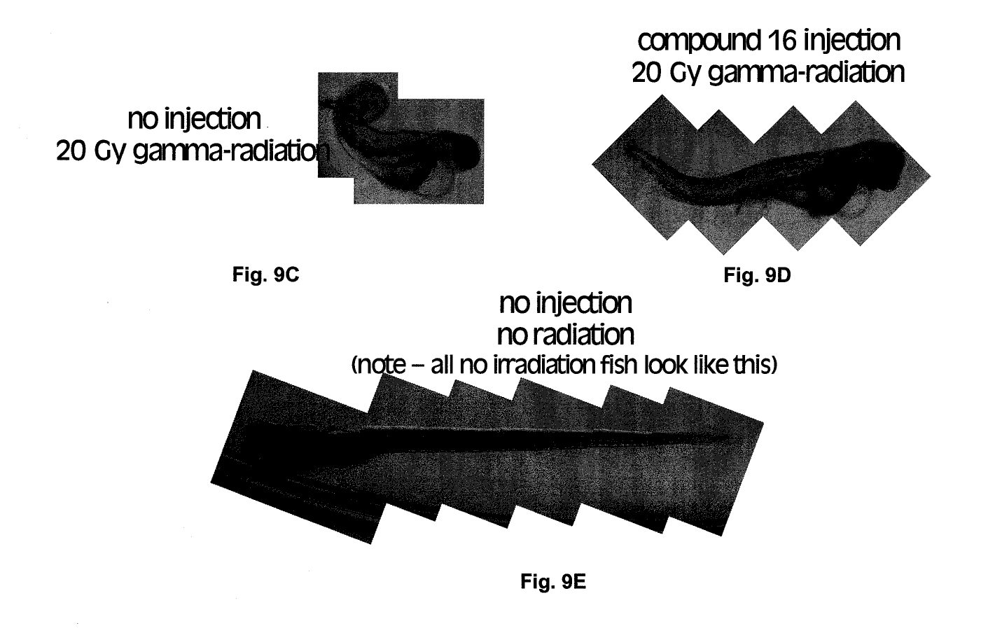

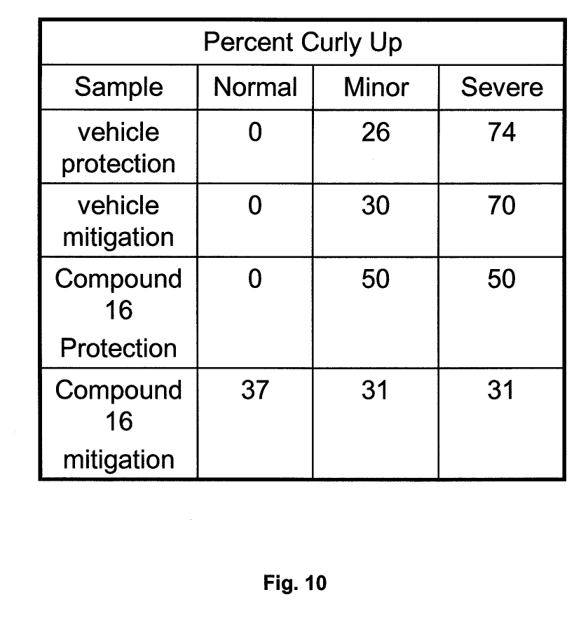

Four days post-fertilization, zebrafish exposed to radiation only (20 Gy), show a severe curly up (FIG. 9A). At the same time point, zebrafish exposed to radiation and injected with compound 16, show no curly up (FIG. 9B). Six days post fertilization, the zebrafish exposed to radiation only (20 Gy) are severely curled up (FIG. 9C), while the zebrafish exposed to radiation and injected with compound 16 show a minor curly up phenotype (FIG. 9D). The non-irradiated and non-injected control fish were straight and showed no bending, or curly up (FIG. 9E). The results of the protection assay for compound 16 caused 50 of the embryos to have minor curly up and 50 to be classified as severe curly up, versus 26 and 74, respectively, for control fish with PBS injection (FIG. 10).

The control embryos for the mitigation assay had similar classifications as for the controls in the protection assay. The mitigation assay results for compound 16 actually show better results than the protection assay: 37 embryos were classified as normal with no bending, 31 with minor curly up, and 31 with severe curly up (FIG. 10). This result substantiates the fact that compound 16 displays radiation mitigation properties in vivo. The images shown were consistent with all embryos and are of different fish. The degree of curly up did not progress over time.

### Mouse Study

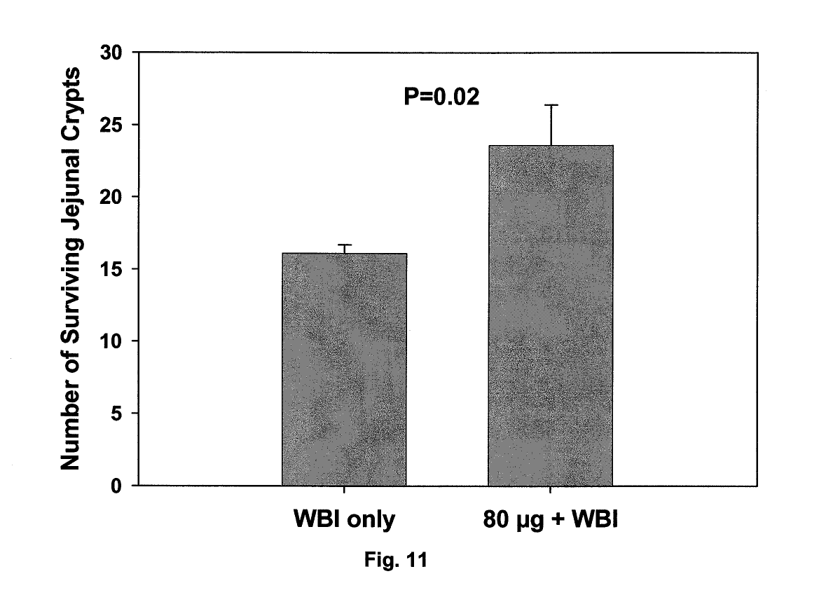

There are well developed clonal assays using mice as a means of assessing radiation effects on normal tissues in vivo. The viability of crypt stem cells in the jejunum of mice was used to determine the amount of damage caused by radiation. In a typical experiment, mice were injected with compound 13 solution 30 min prior to a single dose of whole body irradiation (WBI), ranging from 10 to 25 Gy. These doses are known to produce classical gastrointestinal syndrome in mice. 3.5 days after irradiation the mice are sacrificed and the jejunum was prepared for histological examination. The numbers of regenerating crypts in the jejunal cross-section were counted microscopically at 100×. The resulting number of viable crypt cells was compared to that of irradiated mice that had not been given compound 13. An increase of 47% of surviving crypts was found using compound 13 (FIG. 11). The dose of compound 13 was 5000 times lower than the optimal protective dose of Amifostine (WR-2721), a compound currently in use for treatment of radiation poisoning, [see for example, Pamujula, S.; Graves, R. A.; Freeman, T.; Srinivasan, V.; Bostanian, L. A.; Kishore, V.; Mandal, T. K., “Oral delivery of spray dried PLGA/amifostine nanoparticles,” Journal of Pharmacy and Pharmacology, 2004, 56, 1119-1125.] that provided protection in radiation studies on mice.

From the foregoing detailed description of specific embodiments of the invention, it should be apparent that carbon nanotubes and radical scavenging-carbon nanotube conjugate compositions and methods of using the same are useful in protection from the harmful effects of radiation exposure. Many other types of radical scavenging moieties could be attached to the carbon nanotube using similar protocols outlined herein. Furthermore, one skilled in the art may recognize the ability to freely substitute DWNTs and MWNTs for SWNTs.

Although specific embodiments of the invention have been disclosed herein in some detail, this has been done solely for the purposes of describing various features and aspects of the invention, and is not intended to be limiting with respect to the scope of the invention. It is contemplated that various substitutions, alterations, and/or modifications, including but not limited to those implementation variations which may have been suggested herein, may be made to the disclosed embodiments without departing from the spirit and scope of the invention as defined by the appended claims which follow. Moreover, the radical scavenging mechanism of protection and mitigation is merely a working hypothesis for the observed efficacy of these types of therapeutic agents. There might be different or additional mechanisms whereby these carbon nanotube therapeutics are operating to produce the results described herein.

We claim the following:

1-20. (canceled)

21. A composition comprising:
a) carbon nanotubes covalently derivatized with a plurality of polymer chains; and
b) molecules sequestered within the plurality of polymer chains.

22. The composition of claim 21, wherein polymer chains comprise poly(ethylene glycol).

23. The composition of claim 21, wherein the polymer chains comprise poly(ethylene imine).

24. The composition of claim 21, wherein the carbon nanotubes are selected from the group consisting of single-wall carbon nanotubes, double-wall carbon nanotubes, multi-wall carbon nanotubes and combinations thereof.

25. The composition of claim 21, wherein the sequestered molecules are further associated with the carbon nanotubes by acid-base interactions.

26. The composition of claim 21, wherein the sequestered molecules are further associated with the carbon nanotubes by covalent bonding.

27. The composition of claim 21, wherein the sequestered molecules are further associated with the carbon nanotubes by pi-pi interactions.

28. The composition of claim 21, wherein the sequestered molecules are further associated with the carbon nanotubes by van der Waals interactions.

29. The composition of claim 21, wherein the carbon nanotubes are functionalized with a plurality of carboxylic acid groups and each of the plurality of polymer chains is covalently derivatized to the carbon nanotubes through a carboxylic acid group.

30. The composition of claim 21, wherein the composition is water soluble.

31. A water-soluble composition comprising:
a) carbon nanotubes covalently derivatized with a plurality of poly(ethylene glycol) chains; and
b) molecules sequestered within the plurality of poly(ethylene glycol) chains.

32. The composition of claim 31, wherein the carbon nanotubes are selected from the group consisting of single-wall carbon nanotubes, double-wall carbon nanotubes, multi-wall carbon nanotubes and combinations thereof.

33. The composition of claim 31, wherein the sequestered molecules are further associated with the carbon nanotubes by acid-base interactions.

34. The composition of claim 31, wherein the sequestered molecules are further associated with the carbon nanotubes by covalent bonding.

35. The composition of claim 31, wherein the carbon nanotubes are functionalized with a plurality of carboxylic acid groups.

36. A water-soluble composition comprising:
a) carbon nanotubes covalently derivatized with a plurality of poly(ethylene glycol) chains;
wherein the carbon nanotubes comprise a plurality of carboxylic acid groups; and
wherein each of the plurality of poly(ethylene glycol) chains are covalently derivatized to the carbon nanotubes through a carboxylic acid group; and
b) molecules sequestered within the plurality of poly(ethylene glycol) chains.

37. The composition of claim 36, wherein the carbon nanotubes are selected from the group consisting of single-wall carbon nanotubes, double-wall carbon nanotubes, multi-wall carbon nanotubes and combinations thereof.

38. The composition of claim 36, wherein the sequestered molecules are further associated with the carbon nanotubes by acid-base interactions.

39. The composition of claim 36, wherein the sequestered molecules are further associated with the carbon nanotubes by covalent bonding.
